(19) United States
(12) Patent Application Publication
(10) Pub. No.: US 2003/0225044 A1
(43) Pub. Date: Dec. 4, 2003

(54) Method to increase serum levels of vitamins in animals including humans

(76) Inventors: Stephen E. Bachman, Amarillo, TX (US); Michael E. Hubbert, Arroyo Seco, NM (US)

Correspondence Address: LAW OFFICE OF DALE F. REGELMAN, P.C., 4231 S. Fremont Avenue, Tucson, AZ, 85714, US

(21) Appl. No.: 10/162,111

(22) Filed: Jun. 3, 2002

(51) Int. Cl. A61K 31/59; A61K 31/355; A61K 31/07

(52) U.S. Cl. 514/167; 514/168; 514/458; 514/725

(57) ABSTRACT

A method to increase blood levels in animals, including humans, of one or more vitamins by administering those one or more vitamins to the animal's nasal pharynx, such that the blood levels of each of those one or more vitamins increases throughout about a 6 hour period post-dosing, and optionally, throughout a 24 hour period post-dosing. A method to increase blood levels in animals, including humans, of one or more vitamins by administering those one or more vitamins to the animal's buccal cavity, such that the blood levels of each of those one or more vitamins increases throughout about a 6 hour period post-dosing, and optionally, throughout a 24 hour period post-dosing.

### FIELD OF THE INVENTION

The present invention relates to a method and means of increasing blood levels of one or more vitamins by administering those one or more vitamins, singly or in combination with one or more carriers, to the nasal pharynx, or to the buccal cavity, of animals, including humans.

### BACKGROUND OF THE INVENTION

Nutrients, including vitamins, and various medicaments have traditionally been administered orally or by injection (subcutaneous, intramuscular) to animals, including humans. In the context of feedstock animals, i.e. meat-producing animals, such nutrients/medicaments have traditionally been added to the animals food and/or water. Such oral administration, however, does not effectively deliver the proper dosage to each and every animal. Significantly, animals that are sick often do not eat or drink properly. These sick animals, however, are in greatest need of vitamins, nutrients, and/or medicaments.

Administration of vitamins, nutrients, and/or medicaments to feedstock animals via intramuscular injection is an effective, but undesirable route of dosing. This route requires sterile procedures that can be difficult to maintain under field conditions. Chemical compounds can be painful and solubility of the active ingredient can cause formulation problems. Many times these formulations do not flow at colder temperatures and become very difficult to administer accurate doses.

Intramuscular injections often result in tissue bruising, injection site lesions and concomitant product loss post-mortem. Subcutaneous injection can be difficult to administer and can cause swelling at the injection site. Furthermore, subcutaneous injections may be given intramuscularly by mistake and reduce the effectiveness of the active compound. Animals/humans do not like injections and can move during the administration causing the needle to break off at the injection site. This creates a hazard for the animal/human and a contaminant in the food chain.

Modernly, certain nutrients and/or medicaments have been administered to feedstock animals using transdermal techniques/formulations. Not all nutrients and/or medicaments, however, can be effectively administered transdermally. In addition, transdermal administration often requires preliminary skin preparation, i.e. shaving, and can be hazardous to application personnel. Depending on the viscosity of the transdermal formulation, a certain amount of the transdermal formulation is shaken off the animal, and/or affected by environmental conditions, and therefore, never enters the animal's blood stream.

What is needed is a method to administer one or more vitamins to animals, including humans, where that method is easy to perform, cost-effective, and time-effective. In addition, what is needed is a method to administer one or more vitamins to feedstock animals such that the blood levels of those one or more vitamins increases over a six hour period post-dosing, and in certain embodiments, over a twenty-four hour period post-dosing.

### SUMMARY OF THE INVENTION

Applicants' invention includes a method to increase blood levels of one or more vitamins such that the blood levels of those one or more vitamins increases over the six hour period after administration. In certain embodiments, Applicants' method administers at a first time a first vitamin to an animal via the buccal cavity, i.e. the mouth, where that animal has a first blood level of the first vitamin just prior to dosing. Using Applicants' method a second blood level of the first vitamin is obtained at a second time, six hours post-dosing for example, where that second blood level is greater than the first blood level.

In certain embodiments, Applicants' method administers at a first time a first vitamin to an animal via the nasal pharynx, where that animal has a first blood level of the first vitamin just prior to dosing. Using Applicants' method a second blood level of the first vitamin is obtained at a second time, six hours post-dosing for example, where that second blood level is greater than the first blood level. In certain embodiments, a third blood level of that first vitamin is obtained at a third time, twenty-four hours post-dosing for example, where that third blood level is greater than the second blood level. Vitamins effectively administered via the buccal cavity/nasal pharynx using Applicants' method include, without limitation, Vitamin D, Vitamin E, Vitamin A, and combinations thereof.

### BRIEF DESCRIPTION OF THE DRAWINGS

The invention will be better understood from a reading of the following detailed description taken in conjunction with the drawings in which like reference designators are used to designate like elements, and in which:

FIG. 1 is a graph showing blood levels of 25-Hydroxy-Vitamin D₃ at 6 and 24 hours following intravenous administration;

FIG. 2 is a graph showing blood levels of 25-Hydroxy-Vitamin D₃ at 6 and 24 hours following nasal delivery of various 25-Hydroxy-Vitamin D₃-containing formulations;

FIG. 3 is a graph showing blood levels of 25-Hydroxy-Vitamin D₃ at 6 hours post-dosing for 4 different dosages;

FIG. 4 is a chart showing the change in blood levels of 25-Hydroxy-Vitamin D₃ 6 hours after administration for 4 different dosings;

FIG. 5 is a chart showing the change 6 hours post-dosing in serum levels of 25-Hydroxy-Vitamin D₃ per milligram administered;

FIG. 6 is a graph showing serum levels of 25-Hydroxy-Vitamin D₃ 6 hours post-dosing for nasal and buccal administrations;

FIG. 7 is a chart showing the change in serum levels of 25-Hydroxy-Vitamin D₃ per milligram administered for nasal and buccal administrations;

FIG. 8 is a graph showing blood levels of Vitamin A at 6 and 24 hours following nasal delivery of various Vitamin A-containing formulations; and

FIG. 9 is a graph showing blood levels of Vitamin E at 6 and 24 hours following nasal delivery of various Vitamin E-containing formulations.

### DETAILED DESCRIPTION OF THE PREFERRED EMBODIMENTS

Applicants' invention will be described as embodied in a method to increase serum levels of one or more vitamins in feedstock animals. The following description of Applicant's nasal delivery composition and method is not meant, however, to limit Applicant's invention to administering vitamins to meat-producing animals, as the invention herein can be applied generally to administering one or more vitamins, nutrients, and/or medicaments to animals, including humans, such that the serum levels of those one or more vitamins, nutrients, and/or medicaments increases over a six hour period, and optionally, over a twenty-four hour period.

### Vitamin D

Applicants have found that administration of Vitamin D via delivery to the nasal pharynx of animals, including humans, results in an increase of serum Vitamin D levels in the 6 hour period following administration. In certain embodiments of Applicants' invention, the serum Vitamin D level increases in the 24 hour period following administration. In certain embodiments, the serum Vitamin D level at 24 hours exceeds the serum Vitamin D level at 6 hours. By “Vitamin D,” Applicants mean Ergosterol, Compound I, and the known derivatives and analogs of Compound I, including without limitation Compounds II, III, IV, V, and VI.

Vitamin D is a steroid hormone that functions to regulate specific gene expression following interaction with its intracellular receptor. The biologically active form of the hormone is 1,25-dihydroxy vitamin D₃, i.e. Compound VI-also termed calcitriol. Calcitriol functions primarily to regulate calcium and phosphorous homeostasis.

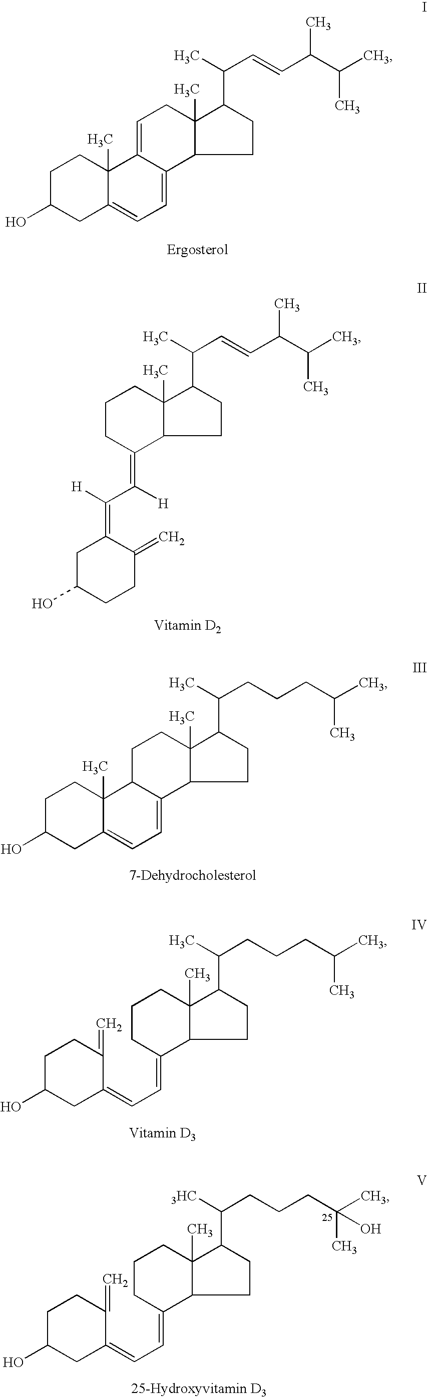

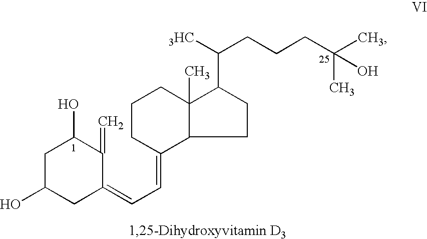

Active calcitriol is derived from ergosterol (produced in plants) and from 7-dehydrocholesterol (produced in the skin). Ergocalciferol (Vitamin D₂) is formed by ultraviolet irradiation of ergosterol. In the skin 7-dehydrocholesterol is converted to cholecalciferol (Vitamin D₃) following ultraviolet irradiation.

Vitamin D₂ and D₃ are processed to D₂-calcitriol and D₃-calcitriol, respectively, by the same enzymatic pathways in the body. Cholecalciferol (or egrocalciferol) are absorbed from the intestine and transported to the liver bound to a specific vitamin D-binding protein. In the liver cholecalciferol is hydroxylated at the 25 position by a specific D₃-25-hydroxylase generating 25-hydroxy-D₃ [25-(OH)D₃] which is the major circulating form of vitamin D. Conversion of 25-(OH)D₃ to its biologically active form, calcitriol, occurs through the activity of a specific D₃-1-hydroxylase present in the proximal convoluted tubules of the kidneys, and in bone and placenta. 25-(OH)D₃ can also be hydroxylated at the 24 position by a specific D₃-24-hydroxylase in the kidneys, intestine, placenta and cartilage.

Calcitriol functions in concert with parathyroid hormone (PTH) and calcitonin to regulate serum calcium and phosphorous levels. PTH is released in response to low serum calcium and induces the production of calcitriol. In contrast, reduced levels of PTH stimulate synthesis of the inactive 24,25-(OH)₂D₃. In the intestinal epithelium, calcitriol functions as a steroid hormone in inducing the expression of calbindinD28K, a protein involved in intestinal calcium absorption. The increased absorption of calcium ions requires concomitant absorption of a negatively charged counter ion to maintain electrical neutrality. The predominant counter ion is Pi. When plasma calcium levels fall the major sites of action of calcitriol and PTH are bone where they stimulate bone resorption and the kidneys where they inhibit calcium excretion by stimulating reabsorption by the distal tubules. The role of calcitonin in calcium homeostasis is to decrease elevated serum calcium levels by inhibiting bone resorption.

The main symptom of vitamin D deficiency in children is rickets and in adults is osteomalacia. Rickets is characterized improper mineralization during the development of the bones resulting in soft bones. Osteomalacia is characterized by demineralization of previously formed bone leading to increased softness and susceptibility to fracture.

If the body does not get enough vitamin D and calcium, the person/animal is at higher risk for bone mass loss known as osteoporosis. Low levels of vitamin D also increases the risk of bone softening, known as osteomalacia, in older adults. Children with a significant vitamin D deficiency may develop rickets, or defective bone growth. Administering Vitamin D derivative/analogs by nasal administration provides a simple, economical, and efficient procedure to assure proper nutritional levels of these vitamins.

In addition to administering Vitamin D and/or Vitamin D derivatives and analogs for proper health and nutrition, increasing blood levels of Vitamin D and/or Vitamin D derivative and analogs in feedstock animals increases meat tenderness postmortem. Recent research has indicated that skeletal muscle is an important target organ for Vitamin D₃. Vitamin D₃ has been shown to increase uptake and transport of calcium by the mitochondrial and sarcoplasmic membranes of rachitic chick skeletal muscle. In addition, 1,25-dihydroxyvitamin D and 25-hydroxyvitamin D have been reported to increase calcium uptake and cyclic AMP levels, and stimulate the phosphorylation of several membrane proteins including those which calmodulin binding capacity potentates in skeletal muscle. Vitamin D₃ has been shown to increase the phospholipid composition of the sarcoplasmic reticulum and mitochondria of rachitic chick skeletal muscle. Thus, Vitamin D₃ increased calcium transportation has been related to the Ca⁺⁺ pump and not to increased permeability of the membrane by lowering the Km and increasing the Vmax.

The present invention provides a method for increasing the tenderness of livestock meat tissues, which achieves the desired objectives. It involves increasing the calcium concentration in livestock muscle tissue prior to harvest of the animal to a degree sufficient to activate or enhance postmortem tenderization mechanisms. This is accomplished, most preferably, through the administration of Vitamin D, its analogs or derivatives, or combinations thereof, to livestock at concentrations above those required nutritionally in order to decrease shear force (increase tenderness) of meat tissues. The inventors have found that administering via nasal delivery Vitamin D to meat producing animals, rather than treating the meat with calcium chloride postmortem, is a more effective means of tenderizing meat.

The following examples are presented to further illustrate to persons skilled in the art how to make and use the invention and to identify presently preferred embodiments thereof. These examples are not intended, however, as limitations upon the scope of the invention, which is defined only by the appended claims.

### EXAMPLE I

### Vitamin D

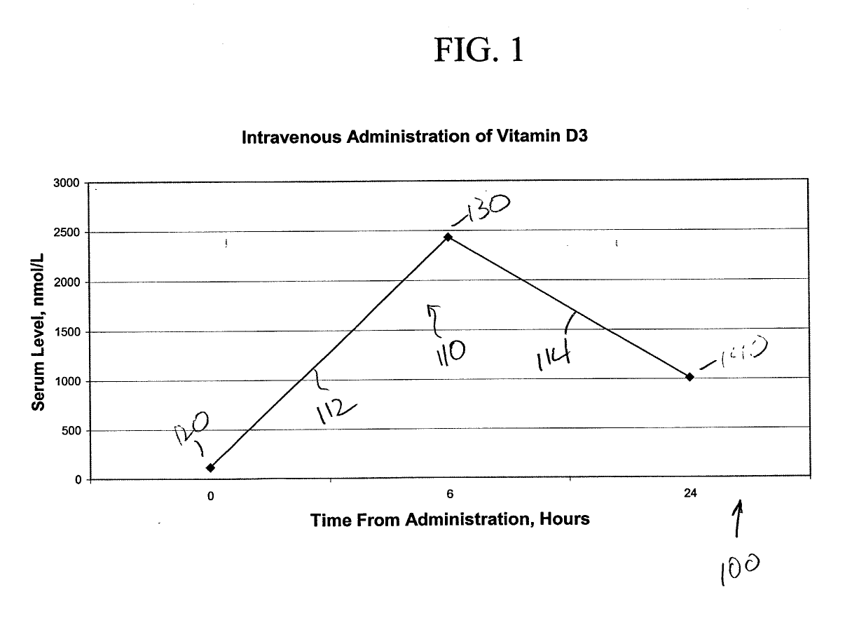

Applicants infused three (3) beef steers of mixed breeding with 125 milligrams of 25-Hydroxy-Vitamin D₃ intravenously. Blood was drawn just prior to the intravenous infusion and at 6 hours and 24 hours post administration. Referring now to FIG. 1, graph 100 includes curve 110 which shows the measured blood levels of 25-Hydroxy-Vitamin D₃ as a function of time.

Point 120, representing 116 nanomoles per liter, comprises the serum level of 25-Hydroxy-Vitamin D₃ just prior to the intravenous infusion. At 6 hours post administration, the serum blood level was measured as 2432 nanomoles per liter, i.e. point 130. Curve portion 112 clearly has a positive slope, meaning that the serum level of 25-Hydroxy-Vitamin D₃ increases from 0 hours to 6 hours post administration. At 24 hours post administration, the serum blood level was measured as 1011 nanomoles per liter, i.e. point 140. Curve portion 114 clearly has a negative slope, meaning that the serum level of 25-Hydroxy-Vitamin D₃ decreases from 6 hours to 24 hours post administration.

When administering Vitamin D and/or Vitamin D derivatives/analogs, it is often medically advantageous to obtain serum levels which remain somewhat constant over a period of time, such as a day. Such relatively constant blood levels facilitates a daily administration of the vitamin. In addition, when administering Vitamin D and/or Vitamin D derivatives/analogs to feedstock animals to increase the tenderness of meat postmortem, it is advantageous to obtain an increased serum level just prior to harvest. As those skilled in the art will appreciate, it is not practical to administer Vitamin D and/or Vitamin D derivatives/analogs to feedstock animals through the feed just prior to harvest. Rather, it is logistically easier to administer such Vitamin D and/or Vitamin D derivatives/analogs nasally just prior to harvest. Additionally, it is advantageous to increase the tenderness of meat without the need to repeatedly supplement the animal's feed with Vitamin D for several days or weeks pre-harvest.

What is needed is a simple and economical method to administer Vitamin D and/or Vitamin D derivatives/analogs to feedstock animals within hours prior to harvest. Applicants' method to administer Vitamin D and/or Vitamin D derivatives/analogs to feedstock animals within hours prior to harvest using nasal delivery results in blood levels of Vitamin D and/or Vitamin D derivatives/analogs that increase throughout the next 6 hours, and optionally, throughout the next 24 hours.

### EXAMPLE II

### Vitamin D

To demonstrate the efficacy of nasal administration of Vitamin D, fifteen (15) beef steers of mixed breeding were used to evaluate five treatments (3 replications). These five treatments were:

| | |
|---|---|
| Treatment 1 | Control-no treatment |
| Treatment 2 | 125 mg 25-OH D3, ethanol, intranasal |
| Treatment 3 | 125 mg 25-OH D3, powder, intranasal |
| Treatment 4 | 125 mg 25-OH D3, corn oil, intranasal |
| Treatment 5 | 125 mg 25-OH D3, transdermal (inner |
| | ear) |

Treatments were applied and cattle were bled via jugular venipuncture at 0, 6, and 24 hours post-dosing.

TABLE 1
Average Serum Levels-25-Hydroxy-Vitamin D₃ (nmol/L).

| | | Time | |
|---|---|---|---|
| Treatment | 0 | 6 Hours | 24 Hours |
| 1 | 93 | 107 | 143 |
| 2 | 75 | 226 | 354 |
| 3 | 83 | 104 | 156 |
| 4 | 94 | 95 | 179 |
| 5 | 74 | 81 | 96 |

TABLE 2
Multiple change from time 0 of serum 25-Hydroxy-Vitamin D₃

| Treatment | 0 | 6 Hours | 24 Hours |
|---|---|---|---|
| 1 | 0 | 1.2 | 1.5 |
| 2 | 0 | 3.0 | 4.7 |
| 3 | 0 | 1.2 | 1.9 |
| 4 | 0 | 1.0 | 1.9 |
| 5 | 0 | 1.1 | 1.3 |

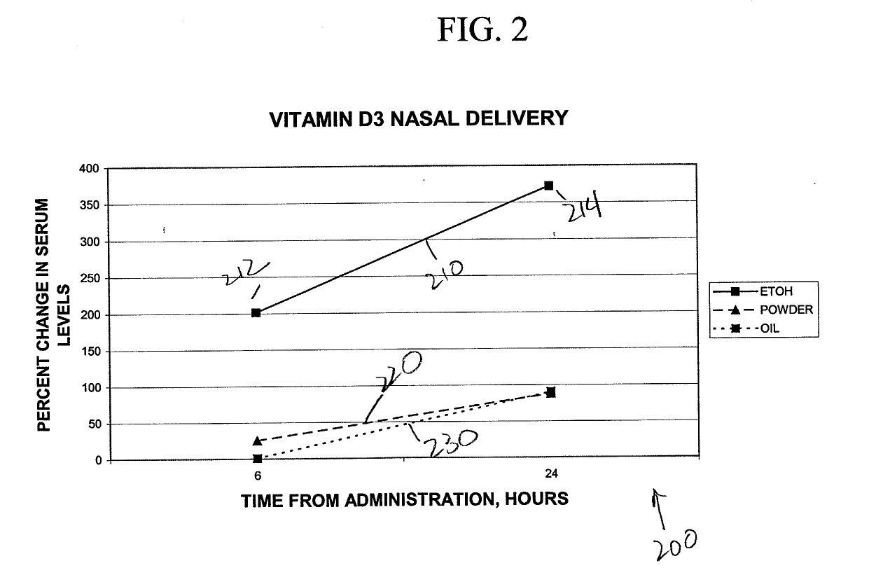

Referring now to FIG. 2, graph 200 shows the percent change in serum 25-Hydroxy-Vitamin D₃ level at 6 and 24 hours post administration. Curve 210 graphically shows the results of Treatment 2, wherein 25-Hydroxy-Vitamin D₃ was dissolved in ethanol, and that ethanolic solution was administered to the animal by nasal delivery. Curve 210 shows that the serum level of 25-Hydroxy-Vitamin D₃ increases throughout the time period from 0 hours to 24 hours post administration using Treatment 2. Point 212 on curve 210 shows more than a three fold increases, i.e. about a 200 percent increase, in the 25-Hydroxy-Vitamin D₃ serum level 6 hours post administration. Point 214 shows a greater than 4.7 fold increase, i.e. about a 375 percent increase, in the 25-Hydroxy-Vitamin D₃ serum level 24 hours post administration.

Curve 220 graphically shows the results of Treatment 3, wherein 25-Hydroxy-Vitamin D₃ powder was administered to the animal by nasal delivery. Curve 220 indicates that the serum level of 25-Hydroxy-Vitamin D₃ increases throughout the time period from 0 hours to 24 hours post administration. Curve 230 graphically shows the results of Treatment 4, wherein 25-Hydroxy-Vitamin D₃ was dissolved in corn oil, and that corn oil solution was administered to the animal by nasal delivery. Curve 230 has a positive slope indicating that the serum level of 25-Hydroxy-Vitamin D₃ increases throughout the time period from 6 hours to 24 hours post administration.

### EXAMPLE III

### Vitamin D

To study the dose/response efficacy of nasal administration of Vitamin D, twelve (12) beef steers of mixed breeding were used to evaluate four treatments (3 replications). These four treatments were:

| | |
|---|---|
| Treatment 1 | Control-no treatment |
| Treatment 2 | 30 mg 25-OH D₃ in 1 ml ethanol, intranasal |
| Treatment 3 | 60 mg 25-OH D₃ in 1 ml ethanol, intranasal |
| Treatment 4 | 90 mg 25-OH D₃ in 1 ml ethanol, intranasal |
| Treatment 5 | 120 mg 25-OH D₃ in 1 ml ethanol, intranasal |

Treatments were applied and cattle were bled via jugular venipuncture at 0 and 6 hours post-dosing.

TABLE 3
Average Serum Levels-25-Hydroxy-Vitamin D₃ (nmoI/L).

| | | Time |
|---|---|---|
| Treatment | 0 | 6 Hours |
| 1 | 133.00 | 134.33 |
| 2 | 121.33 | 149.67 |
| 3 | 144.67 | 184.33 |
| 4 | 131.33 | 180.67 |
| 5 | 122.33 | 191.67 |

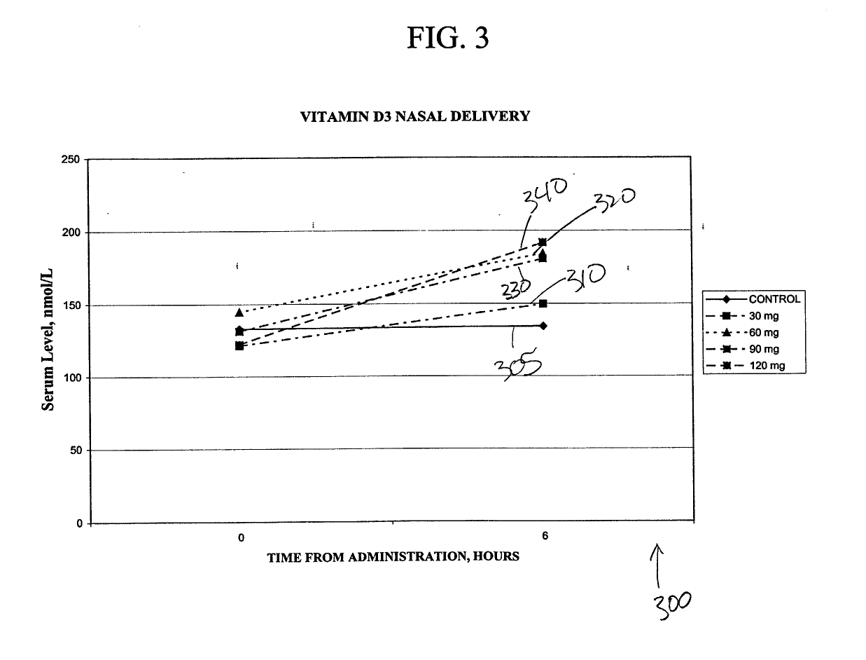

Referring now to FIG. 3, graph 300 shows the data recited in Table 3. Curve 305 represents the data from the Control. As those skilled in the art will appreciate, curve 305 is essentially a flat line having 0 slope. Curve 310 shows the data for Treatment 2, wherein 30 milligrams of 25-Hydroxy-Vitamin D₃ were mixed with 1 milliliter of ethanol, and that one milliliter mixture was delivered to a nostril of the test animals. Curve 320 shows the data for Treatment 3, wherein 60 milligrams of 25-Hydroxy-Vitamin D₃ were mixed with 1 milliliter of ethanol, and that one milliliter mixture was delivered to a nostril of the test animals. Curve 330 shows the data for Treatment 4, wherein 90 milligrams of 25-Hydroxy-Vitamin D₃ were mixed with 1 milliliter of ethanol, and that one milliliter mixture was delivered to a nostril of the test animals. Curve 340 shows the data for Treatment 5, wherein 120 milligrams of 25-Hydroxy-Vitamin D₃ were mixed with 1 milliliter of ethanol, and that one milliliter mixture was delivered to a nostril of the test animals.

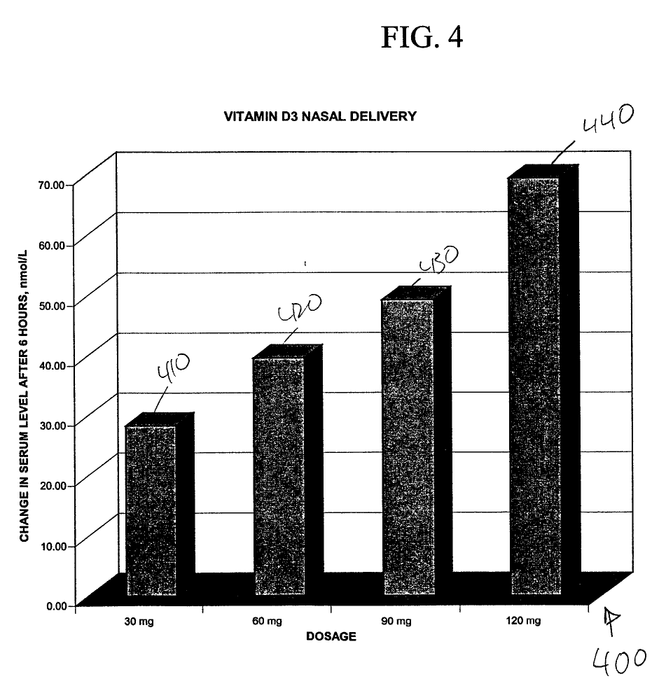

Referring to FIG. 4, chart 400 shows the changes in serum levels of 25-Hydroxy-Vitamin D₃, in nmol/liter, six (6) hours after administration. Bar 410 shows the serum level change for Treatment 2. Bar 420 shows the serum level change for Treatment 3. Bar 430 shows the serum level change for Treatment 4. Bar 440 shows the serum level change for Treatment 5. As expected, the absolute change in serum level increases with increasing dosage.

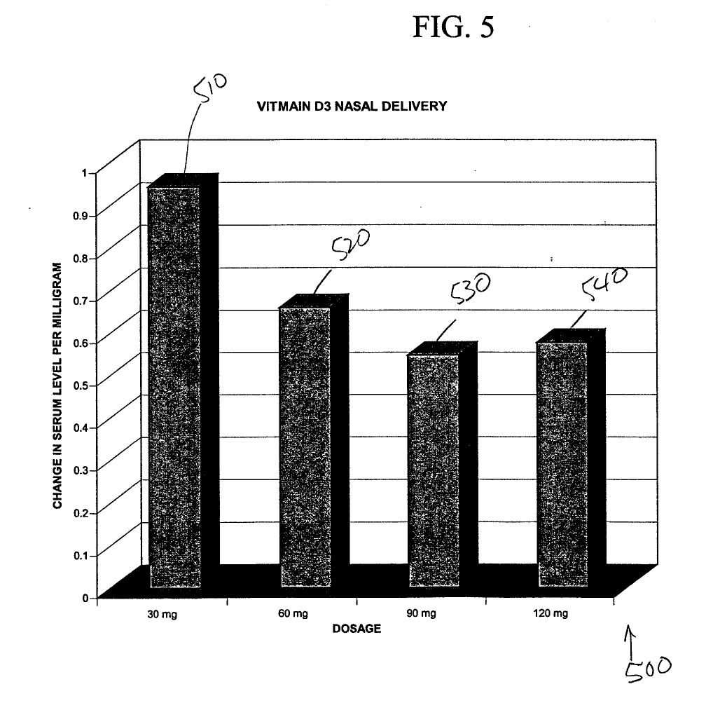

Referring now to FIG. 5, chart 500 shows the change in serum level of 25-Hydroxy-Vitamin D₃ per milligram administered. Bar 510 shows the serum level change, in nmol/L, per milligram administered for Treatment 2. Bar 520 shows the serum level change, in nmol/L, per milligram administered for Treatment 3. Bar 530 shows the serum level change, in nmol/L, per milligram administered for Treatment 4. Bar 540 shows the serum level change, in nmol/L, per milligram administered for Treatment 5.

FIG. 5 shows that Treatment 2, i.e. 30 milligrams of 25-Hydroxy-Vitamin D₃ in 1 milliliter of ethanol gave the maximum change in serum level per milligram administered. The data of Tables 1, 2, and 3, suggests that a dosage of 30 milligrams of 25-Hydroxy-Vitamin D₃ gives the maximum short term serum level change per milligram administered. On the other hand, higher dosages result in increasing serum levels over a greater period of time.

Therefore, the most cost-efficient, readily-administered treatment to give the maximum serum level change per milligram administered six (6) hours post-dosing of 25-Hydroxy-Vitamin D₃ comprises delivering about 30 milligrams of 25-Hydroxy-Vitamin D₃ to the animal by nasal delivery. On the other hand, the most efficient, readily-administered treatment to give increasing serum levels of 25-Hydroxy-Vitamin D₃ more than twenty-four (24) hours after administration comprises Treatment 3 of Example II, above, i.e. 125 milligrams of 25-Hydroxy-Vitamin D₃ in 2 milliliters of ethanol.

### EXAMPLE IV

### Vitamin D

To study the dose/response efficacy of buccal administration of one or more vitamins, beef steers of mixed breeding were used to evaluate three treatments (3 replications). These three treatments were:

| | |
|---|---|
| Treatment 1 | Control-no treatment |
| Treatment 2 | 120 mg 25-OH D₃ in 1 ml ethanol, intranasal |
| Treatment 3 | 120 mg 25-OH D₃ in 1 ml ethanol, buccal |

Treatments were applied and cattle were bled via jugular venipuncture at 0 and 6 hours post-dosing.

TABLE 4
Average Serum Levels-25-Hydroxy-Vitamin D₃ (nmol/L).

| | | Time |
|---|---|---|
| Treatment | 0 | 6 Hours |
| 1 | 133.00 | 134.33 |
| 2 | 122.33 | 191.67 |
| 3 | 82.00 | 158.00 |

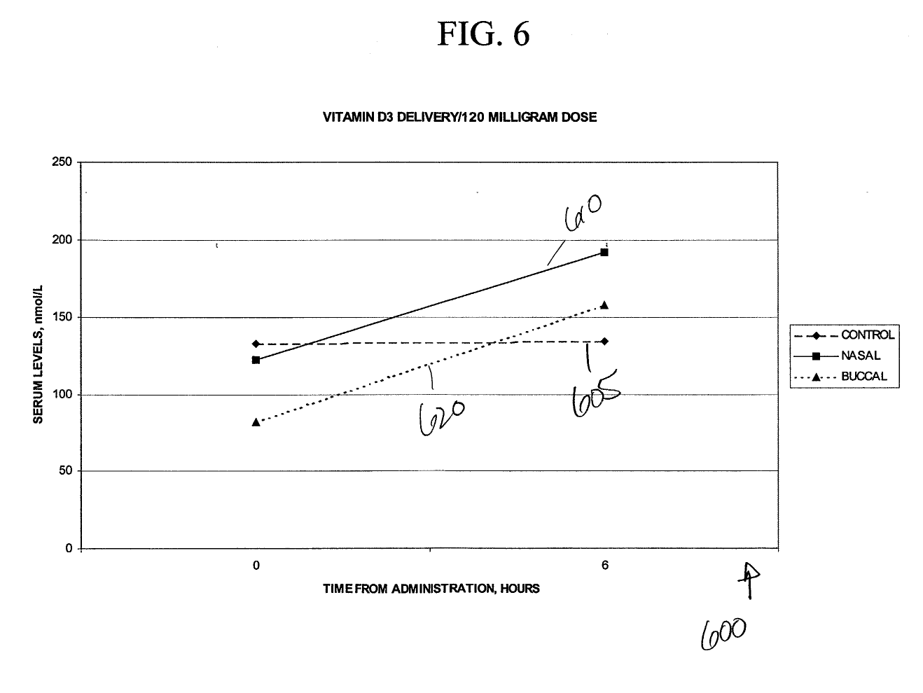

Referring to FIG. 6, graph 600 shows the data of Table 4. Curve 605 shows the data for the control, i.e. no 25-Hydroxy-Vitamin D₃ administered. Curve 610 shows the data for Treatment 2. Treatment 2 of this Example comprises Treatment 5 of Example III. Curve 620 shows the data for Treatment 3 comprising administering 120 milligrams of 25-Hydroxy-Vitamin D₃ in ethanol in the buccal cavity, i.e. the mouth. As those skilled in the art will appreciate, curves 610 and 620 have essentially the same slope. Therefore, buccal administration of about 125 milligrams of 25-Hydroxy-Vitamin D₃ in ethanol is comparatively as effective as nasal administration of 125 milligrams of 25-Hydroxy-Vitamin D₃ in 1 ml of ethanol.

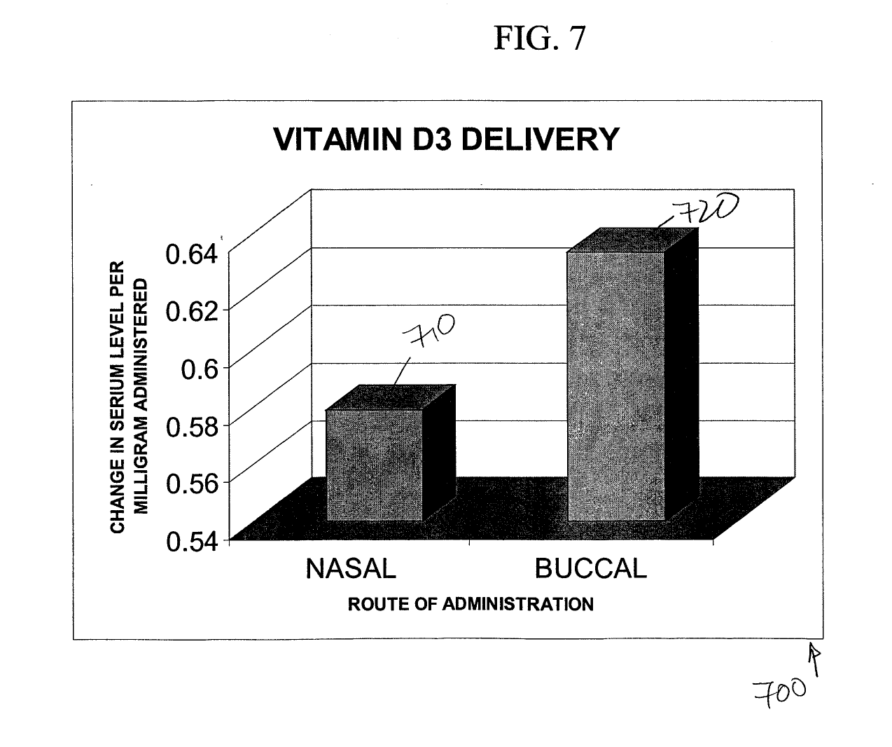

Referring to FIG. 7, chart 7 shows the change in serum level of 25-Hydroxy-Vitamin D₃ after 6 hours for Treatments 2 and 3. Nasal administration showed a serum level change of about 0.57 nmol/L for each milligram administered. Buccal administration showed a serum level change of about 0.63 nmol/L for each milligram administered. Thus, FIG. 7 shows that buccal administration of one or more vitamins may be even more effective that nasal administration of those one or more vitamins.

### Vitamin A

Applicants' have found that administration of Vitamin A via delivery to the nasal passage can result in a short term serum level increase, or alternatively, in a more long term serum level increase. By “Vitamin A,” Applicants mean Vitamin A precursors, including one or more β-carotene, and one or more of four biologically active molecules, including, all-trans retinal (Compound VII), 11-cis-retinal (Compound VIII), retinol (Compound IX), and retinoic acid, (Compound X).

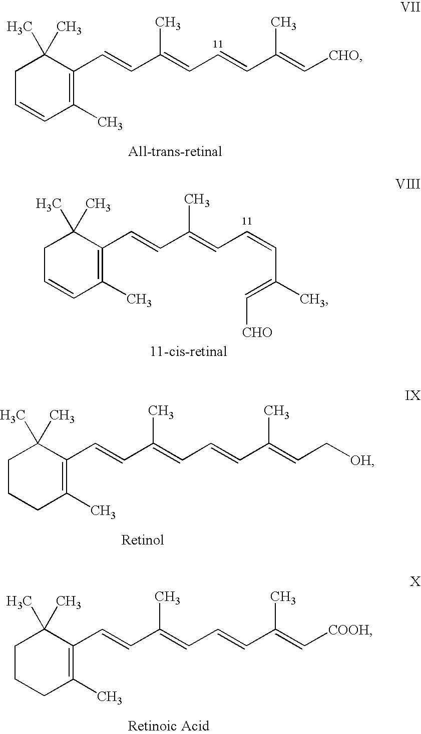

Each of these compounds are derived from the plant precursor molecule, β-carotene (a member of a family of molecules known as carotenoids). Beta-carotene, which consists of two molecules of retinal linked at their aldehyde ends, is also referred to as the provitamin form of vitamin A.

Ingested β-carotene is cleaved in the lumen of the intestine by β-carotene dioxygenase to yield retinal. Retinal is reduced to retinol by retinaldehyde reductase, an NADPH requiring enzyme within the intestines. Retinol is esterified to palmitic acid and delivered to the blood via chylomicrons. The uptake of chylomicron remnants by the liver results in delivery of retinol to this organ for storage as a lipid ester within lipocytes. Transport of retinol from the liver to extrahepatic tissues occurs by binding of hydrolyzed retinol to aporetinol binding protein (RBP). the retinol-RBP complex is then transported to the cell surface within the Golgi and secreted. Within extrahepatic tissues retinol is bound to cellular retinol binding protein (CRBP). Plasma transport of retinoic acid is accomplished by binding to albumin.

Within cells both retinol and retinoic acid bind to specific receptor proteins. Following binding, the receptor-vitamin complex interacts with specific sequences in several genes involved in growth and differentiation and affects expression of these genes. In this capacity retinol and retinoic acid are considered hormones of the steroid/thyroid hormone superfamily of proteins. Vitamin D also acts in a similar capacity. Several genes whose patterns of expression are altered by retinoic acid are involved in the earliest processes of embryogenesis including the differentiation of the three germ layers, organogenesis and limb development.

Photoreception in the eye is the function of two specialized cell types located in the retina; the rod and cone cells. Both rod and cone cells contain a photoreceptor pigment in their membranes. The photosensitive compound of most mammalian eyes is a protein called opsin to which is covalently coupled an aldehyde of vitamin A. The opsin of rod cells is called scotopsin. The photoreceptor of rod cells is specifically called rhodopsin or visual purple. This compound is a complex between scotopsin and the 11-cis-retinal (also called 11-cis-retinene) form of vitamin A. Rhodopsin is a serpentine receptor imbedded in the membrane of the rod cell. Coupling of 11-cis-retinal occurs at three of the transmembrane domains of rhodopsin. Intracellularly, rhodopsin is coupled to a specific G-protein called transducin.

When the rhodopsin is exposed to light it is bleached releasing the 11-cis-retinal from opsin. Absorption of photons by 11-cis-retinal triggers a series of conformational changes on the way to conversion all-trans-retinal. One important conformational intermediate is metarhodopsin II. The release of opsin results in a conformational change in the photoreceptor. This conformational change activates transducin, leading to an increased GTP-binding by the a-subunit of transducin. Binding of GTP releases the a-subunit from the inhibitory b- and g-subunits. The GTP-activated a-subunit in turn activates an associated phosphodiesterase; an enzyme that hydrolyzes cyclic-GMP (cGMP) to GMP. Cyclic GMP is required to maintain the Na⁺ channels of the rod cell in the open conformation. The drop in cGMP concentration results in complete closure of the Na⁺ channels. Metarhodopsin II appears to be responsible for initiating the closure of the channels. The closing of the channels leads to hyperpolarization of the rod cell with concomitant propagation of nerve impulses to the brain.

Retinol also functions in the synthesis of certain glycoproteins and mucopolysaccharides necessary for mucous production and normal growth regulation. This is accomplished by phosphorylation of retinol to retinyl phosphate which then functions similarly to dolichol phosphate.

Vitamin A is stored in the liver and deficiency of the vitamin occurs only after prolonged lack of dietary intake. The earliest symptoms of vitamin A deficiency are night blindness. Additional early symptoms include follicular hyperkeratinosis, increased susceptibility to infection and cancer and anemia equivalent to iron deficient anemia. Prolonged lack of vitamin A leads to deterioration of the eye tissue through progressive keratinization of the cornea, a condition known as xerophthalmia.

The increased risk of cancer in vitamin A deficiency is thought to be the result of a depletion in β-carotene. Beta-carotene is a very effective antioxidant and is suspected to reduce the risk of cancers known to be initiated by the production of free radicals. Of particular interest is the potential benefit of increased β-carotene intake to reduce the risk of lung cancer in smokers. Symptoms of significant deficiency include:

lowered resistance to infections;

problems with reproduction;

poor growth;

improper tooth formation;

rough, dry and pimply skin;

digestive problems;

night blindness; and

eye disease, including xerophthalmia.

The following example is presented to further illustrate to persons skilled in the art how to make and use the invention and to identify a presently preferred embodiment thereof. This examples is not intended, however, as a limitation upon the scope of the invention, which is defined only by the appended claims.

### EXAMPLE V

### Vitamin A

To demonstrate the efficacy of nasal administration of Vitamin A, fifteen (15) beef steers of mixed breeding were used to evaluate five treatments (3 replications). These five treatments were:

| | |
|---|---|
| Treatment 1 | Negative Control-no treatment; |
| Treatment 2 | Positive Control-15 ml drench comprising 1,000,000 |
| | IU of Vitamin A dissolved into 15 ml water; |
| Treatment 3 | Solid form vitamins dissolved into 2 ml ethanol to |
| | provide 1,000,000 IU A, 100,000 IU D, and 1000 IU B |
| | (2 ml dose/animal, 1 ml per nostril); |
| Treatment 4 | 50 g vitamin E oil, 5 g vitamin A oil, 2.25 g vitamin D |
| | oil, 0.25 g Tween 80, 1.0 g nicotinic acid, 0.4 g |
| | Carbopol 934P, 0.2 g Pemulen TR2, 0.25 ml 18% |
| | NaOH, distilled water q.s. Formulation provides |
| | 1,000,000 IU A, 100,000 IU D, and 1000 IU E. (2 ml |
| | dose/animal, 1 ml per nostril) |
| Treatment 5 | 50 g vitamin E oil, 5 g vitamin A oil, 2.25 g vitamin D |
| | oil, 0.25 g Tween 80, 0.3 g Carbopol TR2, .20 ml 18% |
| | NaOH, distilled water q.s. Formulation provides |
| | 1,000,000 IU A, 100,000 IU D, and 1000 IU E. (2 ml |
| | dose/animal, 1 ml per nostril) |

Tween™ 80 is a trademark of ICI Americas, Inc. Carbopol® polymers are used as rheology modifiers, suspending agents and stabilizers. Carbopol® 934 offers stability at high viscosity and produces thick formulations such as heavy gels, emulsions and suspensions. In aqueous systems, Carbopol® 934 exhibits short flow (quick recovery) properties. Carbopol® is a trademark of Noveon, Inc. (formerly The B. F. GOODRICH COMPANY). Pemulen® TR-2 contains a high level of hydrophobic groups and can emulsify a high level of oil (up to 60-80% by weight). Pemulen® is a trademark of Noveon, Inc. (formerly The B. F. GOODRICH COMPANY).

Treatments were applied and cattle were bled via jugular venipuncture at 0, 6, and 24 hours post-dosing. Table 3 summarizes analyses of serum levels of Vitamin A in nanograms per milliliter.

TABLE 5
Average Serum Levels-Vitamin A (ng/ml).

| | | Time | |
|---|---|---|---|
| Treatment | 0 | 6 Hours | 24 Hours |
| 1 | 286 | 278 | 280 |
| 2 | 230 | 277 | 247 |
| 3 | 302 | 344 | 326 |
| 4 | 321 | 314 | 321 |
| 5 | 343 | 349 | 353 |

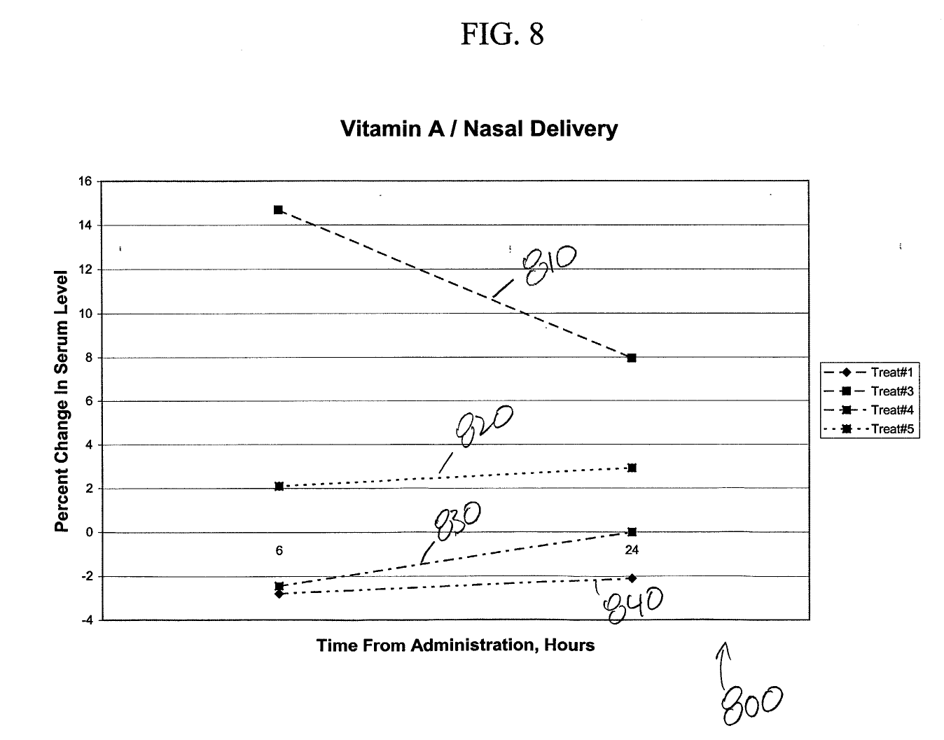

Referring now to FIG. 8, graph 800 graphically depicts the percent change in Vitamin A blood levels as a function of time from dosing. Curve 810 shows the serum Vitamin A levels for Treatment #3, i.e. nasal administration of a Vitamin A/ethanol solution. Curve 810 has a negative slope, showing that the serum level of Vitamin A decreased from 6 to 24 hours post-dosing after an initial increase in the first 6 hours. Thus, if a rapid increase of serum Vitamin A levels is desired, nasal administration of a Vitamin A/ethanol mixture is an easy, economical method to obtain the desired serum levels.

Curve 820 shows the serum Vitamin A levels for Treatment #5. Curve 820 shows that serum levels of Vitamin A increased from 0 hours to 24 hours post-dosing. Curve 830 shows the serum Vitamin A levels for Treatment #4. Curve 820 has a positive slope. Thus using Treatment #4 the serum levels of Vitamin A increased continuously from 6 hours to 24 hours post-dosing. Thus, if a prolonged Vitamin A serum level is desired, nasal administration of Vitamin A using either Treatment #4 or Treatment #5 is an easy, economical method to obtain the desired serum levels.

### Vitamin E

Applicants' have found that administration of Vitamin E via delivery to the nasal passage can result in a prolonged serum level increase. By “Vitamin E,” Applicants mean a mixture of several related compounds known as tocopherols. The α-tocopherol molecule is the most potent of the tocopherols. Vitamin E is absorbed from the intestines packaged in chylomicrons. It is delivered to the tissues via chylomicron transport and then to the liver through chylomicron remnant uptake. The liver can export vitamin E in VLDLs. Due to its lipophilic nature, vitamin E accumulates in cellular membranes, fat deposits and other circulating lipoproteins. The major site of vitamin E storage is in adipose tissue.

The major function of vitamin E is to act as a natural antioxidant by scavenging free radicals and molecular oxygen. In particular vitamin E is important for preventing peroxidation of polyunsaturated membrane fatty acids. The vitamins E and C are interrelated in their antioxidant capabilities. Active α-tocopherol can be regenerated by interaction with Vitamin C following scavenge of a peroxy free radical. Alternatively, α-tocopherol can scavenge two peroxy free radicals and then be conjugated to glucuronate for excretion in the bile.

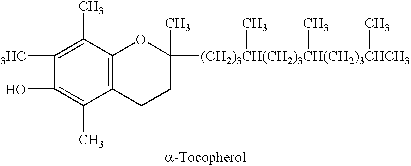

The major symptom of vitamin E deficiency in humans is an increase in red blood cell fragility. Since vitamin E is absorbed from the intestines in chylomicrons, any fat malabsorption diseases can lead to deficiencies in vitamin E intake. Neurological disorders have been associated with vitamin E deficiencies associated with fat malabsorptive disorders. Increased intake of vitamin E is recommended in premature infants fed formulas that are low in the vitamin as well as in persons consuming a diet high in polyunsaturated fatty acids. Polyunsaturated fatty acids tend to form free radicals upon exposure to oxygen and this may lead to an increased risk of certain cancers.

Vitamin E is an important antioxidant. Antioxidants protect cells from oxidation. Oxidation can lead to cell damage. Cell damage can lead to chronic health problems, such as heart disease and cancer. Vitamin E works closely with other antioxidants, like Vitamin C and selenium, to help protect the body. Vitamin E improves the way the body uses Vitamin A. It may help protect against ion the toxic effects of some metals, such as lead.

The following example is presented to further illustrate to persons skilled in the art how to make and use the invention and to identify a presently preferred embodiment thereof. This examples is not intended, however, as a limitation upon the scope of the invention, which is defined only by the appended claims.

### EXAMPLE VI

### Vitamin E

To demonstrate the efficacy of nasal administration of Vitamin A, fifteen (15) beef steers of mixed breeding were used to evaluate five treatments (3 replications). These five treatments were:

| | |
|---|---|
| Treatment 1 | Negative Control-no treatment |
| Treatment 2 | Positive Control-15 ml drench with 1200 IU of |
| | Vitamin E dissolved into 15 ml water; |
| Treatment 3 | Solid form vitamins dissolved into 2 ml ethanol to |
| | provide 1,000,000 IU A, 100,000 IU D, and 1000 |
| | IU E (2 ml dose/animal, 1 ml per nostril); |
| Treatment 4 | 50 g vitamin E oil, 5 g vitamin A oil, 2.25 g |
| | vitamin D oil, 0.25 g Tween 80, 1.0 g nicotinic |
| | acid, 0.4 g Carbopol 934P, 0.2 g Pemulen TR2, .25 |
| | ml 18% NaOH, distilled water q.s. Formulation |
| | provides 1,000,000 IU A, 100,000 IU D, and 1000 |
| | IU E. (2 ml dose/animal, 1 ml per nostril) |
| Treatment 5 | 50 g vitamin E oil, 5 g vitamin A oil, 2.25 g |
| | vitamin D oil, 0.25 g Tween 80, 0.3 g Pemulen |
| | TR2, .20 ml 18% NaOH, distilled water q.s. |
| | Formulation provides 1,000,000 IU A, 100,000 IU |
| | D, and 1000 IU E. (2 ml dose/animal, 1 ml per |
| | nostril) |

Treatments were applied and cattle were bled via jugular venipuncture at 0, 6, and 24 hours post-dosing. Table 4 summarizes analyses of serum levels of Vitamin E in micrograms per milliliter.

TABLE 6
Average Serum Level - Vitamin E (micrograms/ml)

| | | Time | |
|---|---|---|---|
| Treatment | 0 | 6 Hours | 24 Hours |
| 1 | 2.3 | 2.3 | 2.3 |
| 2 | 1.8 | 1.8 | 2.0 |
| 3 | 2.7 | 2.7 | 2.8 |
| 4 | 2.7 | 2.5 | 2.7 |
| 5 | 2.1 | 2.0 | 2.2 |

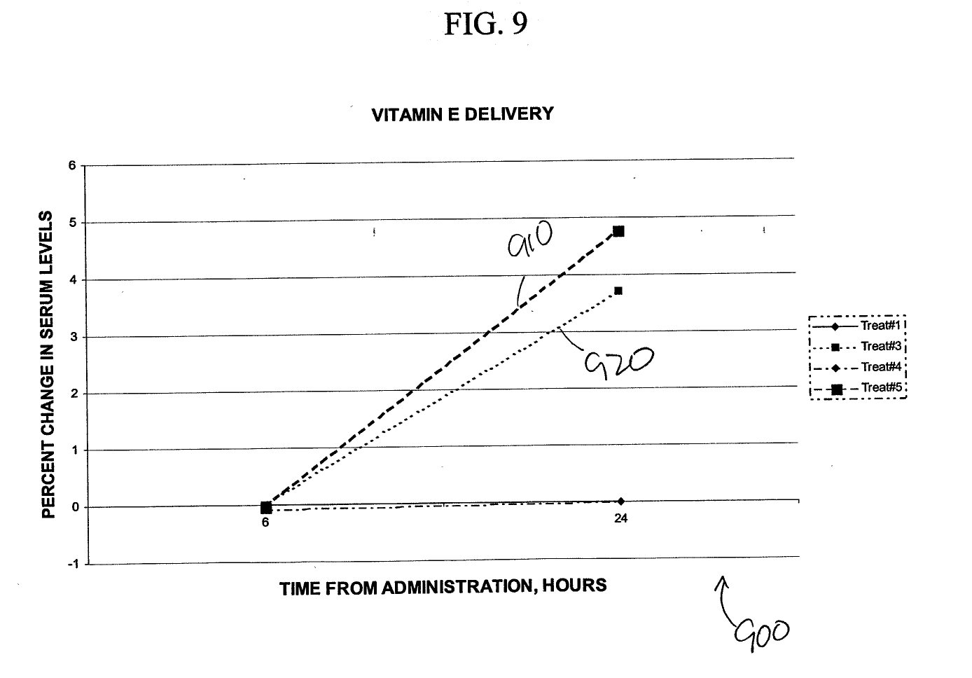

Referring now to FIG. 9, graph 900 recites curves showing the percent change in serum Vitamin E levels as function of time after nasal delivery. Curve 910 shows the serum Vitamin E levels for Treatment #5. Curve 910 has a positive slope. Thus using Treatment #5 the serum levels of Vitamin E increased from 6 hours to 24 hours post-dosing. Curve 920 shows the serum Vitamin E levels using Treatment #3. Curve 920 has a positive slope. Thus using Treatment #3 the serum levels of Vitamin E increased from 6 hours to 24 hours post-dosing. Thus, if a prolonged Vitamin E serum level is desired, nasal administration of Vitamin E using either Treatment #3 or Treatment #5 is an easy, economical method to obtain the desired serum levels.

While the preferred embodiments of the present invention have been illustrated in detail, it should be apparent that modifications and adaptations to those embodiments may occur to one skilled in the art without departing from the scope of the present invention as set forth in the following claims.

We claim:

1. A method to increase blood levels in animals, including humans, of one or more vitamins, comprising the steps of:
administering at a first time a first vitamin to said animal via the nasal pharynx, wherein said animal has a first blood level of said first vitamin at said first time;
obtaining a second blood level of said first vitamin at a second time, wherein said second blood level is greater than said first blood level, and wherein the difference between said second time and said first time is about 6 hours.

2. The method of claim 1, wherein said first vitamin is Vitamin D.

3. The method of claim 2, wherein said first vitamin is selected from the group consisting of Vitamin D₃, 25-Hydroxy-Vitamin D₃, and combinations thereof.

4. The method of claim 3, wherein said first vitamin is 25-Hydroxy-Vitamin D₃.

5. The method of claim 4, further comprising the step of dissolving said 25-Hydroxy-Vitamin D₃ in ethanol.

6. The method of claim 5, further comprising the step of dissolving between about 30 milligrams of said 25-Hydroxy-Vitamin D₃ in ethanol.

7. The method of claim 6, further comprising the step of dissolving about 30 milligrams of 25-Hydroxy-Vitamin D₃ in about 1 milliliter of ethanol.

8. The method of claim 7, wherein the difference between said second blood level and said first blood level is greater than about 0.90 nmol/L per milligram of Vitamin D administered.

9. The method of claim 5, further comprising the step of dissolving about 125 milligrams of 25-Hydroxy-Vitamin D₃ in about 2 milliliters of ethanol.

10. The method of claim 9, wherein said second blood level is about 3 times said first blood level.

11. The method of claim 1, further comprising the step of obtaining a third blood level of said first vitamin at a third time, wherein said third blood level is greater than said second blood level, and wherein the difference between said third time and said second time is about 24 hours.

12. The method of claim 11, further comprising the step of dissolving about 125 milligrams of 25-Hydroxy-Vitamin D₃ in about 2 milliliters of ethanol.

13. The method of claim 12, wherein said third blood level is about 4.7 times said first blood level.

14. The method of claim 13, wherein said third blood level is greater than about 1.5 times said second blood level.

15. The method of claim 11, wherein said first vitamin comprises Vitamin E.

16. The method of claim 15, wherein further comprising the step of dissolving said Vitamin E in ethanol.

17. The method of claim 16, wherein said third blood level is more than 4 percent greater than said second blood level.

18. The method of claim 11, wherein said first vitamin comprises Vitamin A.

19. The method of claim 11, further comprising the steps of:
administering at said first time a second vitamin to said animal via the nasal pharynx, wherein said animal has a fourth blood level of said second vitamin at said first time;
obtaining a fifth blood level of said second vitamin at said second time, wherein said fifth blood level is greater than said fourth blood level, and wherein the difference between said second time and said first time is about 6 hours;
obtaining a sixth blood level of said second vitamin at said third time, wherein said sixth blood level is greater than said fifth level, and wherein the difference between said second time and said third time is about 24 hours.

20. The method of claim 19, wherein said first vitamin is selected from the group consisting of Vitamin D, Vitamin A, and Vitamin E.

21. The method of claim 20, wherein said second vitamin is selected from the group consisting of Vitamin D, Vitamin A, and Vitamin E, and wherein said first vitamin differs from said second vitamin.

22. The method of claim 19, further comprising the steps of:
administering at said first time a third vitamin to said animal via the nasal pharynx, wherein said animal has a seventh blood level of said second vitamin at said first time;
obtaining an eighth blood level of said third vitamin at said second time, wherein said eighth blood level is greater than said seventh blood level, and wherein the difference between said second time and said first time is about 6 hours;
obtaining a ninth blood level of said third vitamin at said third time, wherein said ninth blood level is greater than said eighth level, and wherein the difference between said second time and said third time is about 24 hours.

23. The method of claim 22, wherein said first vitamin is selected from the group consisting of Vitamin D, Vitamin A, and Vitamin E.

24. The method of claim 23, wherein said second vitamin is selected from the group consisting of Vitamin D, Vitamin A, and Vitamin E, and wherein said first vitamin differs from said second vitamin.

25. The method of claim 24, wherein said third vitamin is selected from the group consisting of Vitamin D, Vitamin A, and Vitamin E, and wherein said third vitamin differs from both said first vitamin and said second vitamin.

26. A method to increase blood levels in animals, including humans, of a first vitamin, and a second vitamin, and a third vitamin, comprising the steps of:
administering at a first time said first vitamin to an animal via the nasal pharynx, wherein said animal has a first blood level of said first vitamin at said first time;
administering at said first time said second vitamin to said animal via the nasal pharynx, wherein said animal has a second blood level of said second vitamin at said first time;
administering at said first time said third vitamin to said animal via the nasal pharynx, wherein said animal has a third blood level of said third vitamin at said first time;
obtaining a fourth blood level of said first vitamin at a second time, wherein said fourth blood level is greater than said first blood level, and wherein the difference between said second time and said first time is about 6 hours;
obtaining a fifth blood level of said second vitamin at said second time, wherein said fifth blood level is greater than said second blood level;
obtaining a sixth blood level of said third vitamin at said second time, wherein said sixth blood level is greater than said third blood level;
obtaining a seventh blood level of said first vitamin at a third time, wherein said seventh blood level is greater than said fourth blood level, and wherein the difference between said third time and said second time is about 24 hours;
obtaining an eighth blood level of said second vitamin at said third time, wherein said eighth blood level is greater than said fifth blood level;
obtaining a ninth blood level of said third vitamin at said third time, wherein said ninth blood level is greater than said sixth blood level;
wherein said first vitamin comprises Vitamin D, and wherein said second vitamin comprises Vitamin E, and wherein said third vitamin comprises Vitamin A.

27. A method to increase blood levels in animals, including humans, of Vitamin D comprising the steps of:
administering at a first time Vitamin D to said animal via the nasal pharynx, wherein said animal has a first blood level of Vitamin D at said first time;
obtaining a second blood level of Vitamin D at a second time, wherein the difference between said second time and said first time is about 6 hours, and wherein said second blood level is greater than said first blood level, and wherein the difference between said second blood level and said first blood level is greater than about 0.90 nmol/L per milligram of Vitamin D administered.

28. A method to increase blood levels in animals, including humans, of Vitamin D comprising the steps of:
administering at a first time Vitamin D to said animal via the nasal pharynx, wherein said animal has a first blood level of Vitamin D at said first time;
obtaining a second blood level of Vitamin D at a second time, wherein the difference between said second time and said first time is about 6 hours, and wherein said second blood level is about 3 times said first blood level;
obtaining a third blood level of Vitamin D at a third time, wherein the difference between said third time and said first time is about 24 hours, and wherein said third blood level is about 4.7 times said first blood level.

29. A method for improving meat tenderness derived from an animal post-mortem, comprising the steps of:
administering to said animal Vitamin D via the nasal pharynx at a first time prior to harvest, wherein said animal has a first blood level of Vitamin D at said first time;
obtaining a second blood level of Vitamin D in said animal just prior to harvest, wherein said second blood level is greater than said first blood level, and wherein the difference between said first time and said harvest is less than about 6 hours.

30. The method of claim 29, wherein said second blood level is about 3 times said first blood level.

31. The method of claim 29, wherein the difference between said second blood level and said first blood level is greater than about 0.90 nmol/L per milligram of Vitamin D administered.

32. The method of claim 29, wherein said Vitamin D comprises 25-Hydroxy-Vitamin D₃.

33. The method of claim 22, further comprising the step of dissolving said 25-Hydroxy-Vitamin D₃ in ethanol.

34. The method of claim 23, further comprising the step of dissolving between about 30 milligrams and about 125 milligrams of 25-Hydroxy-Vitamin D₃ in ethanol.

35. The method of claim 34, further comprising the step of dissolving about 30 milligrams of 25-Hydroxy-Vitamin D₃ in about 1 milliliter of ethanol.

36. The method of claim 34, further comprising the step of dissolving about 125 milligrams of 25-Hydroxy-Vitamin D₃ in about 2 milliliters of ethanol.

37. A method to increase blood levels in animals, including humans, of one or more vitamins, comprising the steps of:
administering at a first time a first vitamin to said animal via the buccal cavity, wherein said animal has a first blood level of said first vitamin at said first time;
obtaining a second blood level of said first vitamin at a second time, wherein said second blood level is greater than said first blood level, and wherein the difference between said second time and said first time is about 6 hours.

38. The method of claim 37, wherein said first vitamin is Vitamin D.

39. The method of claim 38, wherein said first vitamin is selected from the group consisting of Vitamin D₃, 25-Hydroxy-Vitamin D₃, and combinations thereof.

40. The method of claim 39, wherein said first vitamin is 25-Hydroxy-Vitamin D₃.

41. The method of claim 40, further comprising the step of dissolving said 25-Hydroxy-Vitamin D₃ in ethanol.

42. The method of claim 41, further comprising the step of dissolving between about 120 milligrams of said 25-Hydroxy-Vitamin in ethanol.

43. The method of claim 42, further comprising the step of dissolving about 120 milligrams of 25-Hydroxy-Vitamin D₃ in about 1 milliliter of ethanol.

44. The method of claim 7, wherein the difference between said second blood level and said first blood level is about 0.63 nmol/L per milligram of Vitamin D administered.

45. A method for improving meat tenderness derived from an animal post-mortem, comprising the steps of:
administering to said animal Vitamin D via the buccal cavity at a first time prior to harvest, wherein said animal has a first blood level of Vitamin D at said first time;
obtaining a second blood level of Vitamin D in said animal just prior to harvest, wherein said second blood level is greater than said first blood level, and wherein the difference between said first time and said harvest is less than about 6 hours.

46. The method of claim 45, wherein the difference between said second blood level and said first blood level is about 0.63 nmol/L per milligram of Vitamin D administered.

47. The method of claim 45, wherein said Vitamin D comprises 25-Hydroxy-Vitamin D₃.

48. The method of claim 47, further comprising the step of dissolving said 25-Hydroxy-Vitamin D₃ in ethanol.

49. The method of claim 48, further comprising the step of dissolving between about 120 milligrams of 25-Hydroxy-Vitamin D₃ in ethanol.

50. The method of claim 49, further comprising the step of dissolving about 120 milligrams of 25-Hydroxy-Vitamin D₃ in about 1 milliliter of ethanol.
